(19) United States
(12) Patent Application Publication
(10) Pub. No.: US 2024/0371493 A1
(43) Pub. Date: Nov. 7, 2024

(54) FEEDBACK LOOP USING WEARABLE-BASED TACTILE INDICATIONS

(71) Applicant: Oura Health Oy, Oulu (FI)

(72) Inventors: Mari Pauliina Karsikas, Oulu (FI); Heli Tuulia Koskimäki, Oulu (FI); Olli-Pekka Puolitaival, Oulu (FI); Iida Paksuniemi, Oulu (FI); Johanna Leena-Kyllikki Still, Oulu (FI); Tarja Karjalainen, Oulu (FI); Juha-Pekka Syrjälä, Oulu (FI); Marko Uusitalo, Oulu (FI); Ville Tiensuu, Oulu (FI); Petteri Lajunen, Oulu (FI); Veera Lindholm, Oulu (FI); Mika Erkkilä, Oulu (FI)

(21) Appl. No.: 18/645,167

(22) Filed: Apr. 24, 2024

Related U.S. Application Data
(60) Provisional application No. 63/499,444, filed on May 1, 2023.

(51) Int. Cl. G16H 20/30; G06F 1/16; G06F 3/01; G06F 3/16

(52) U.S. Cl. CPC G16H 20/30; G06F 1/163; G06F 3/016; G06F 3/167

(57) ABSTRACT

Methods, systems, and devices for tactile feedback are described. A system may be configured to identify a satisfaction of a trigger event for providing tactile or audible feedback to a user via a wearable device. The system may cause the wearable device to provide the tactile or audible feedback to the user in accordance with a learning feedback loop associated with the user, where the learning feedback loop is configurable to train the user, via the tactile or audible feedback, to learn or perform one or more learning objectives associated with the trigger event. The system may then monitor physiological data collected from the user, an additional user, or both, based on communicating the one or more feedback instructions, and selectively modify one or more parameters of the learning feedback loop based on an evaluation of the physiological data relative to the one or more learning objectives.

### CROSS REFERENCE

The present application for patent claims the benefit of U.S. Provisional Patent Application No. 63/499,444 by KARSIKAS et al., entitled “FEEDBACK LOOP USING WEARABLE-BASED TACTILE INDICATIONS,” filed May 1, 2023, assigned to the assignee hereof and expressly incorporated by reference herein.

### FIELD OF TECHNOLOGY

The following relates to wearable devices and data processing, including techniques for providing conscious or subconscious feedback in accordance with a learning feedback loop using wearable-based tactile indications.

### BACKGROUND

Some wearable devices are configured to collect physiological data from users, and provide feedback regarding the acquired physiological data. Typically, feedback provided by wearable devices is in the form of messages and alerts displayed on a graphical user interface (GUI) of the wearable device or a related user device. However, in order for such feedback messages to be useful, a user may be required to pause what they are doing to view and engage with the feedback, thereby decreasing the probability that the user will actually view and act upon the provided feedback. Moreover, providing feedback via GUIs is counter-productive to people's desire to limit or reduce screen time.

### BRIEF DESCRIPTION OF THE DRAWINGS

FIG. 1 illustrates an example of a system that supports a conscious or subconscious learning feedback loop using wearable-based tactile indications in accordance with aspects of the present disclosure.

FIG. 2 illustrates an example of a system that supports a conscious or subconscious learning feedback loop using wearable-based tactile indications in accordance with aspects of the present disclosure.

FIG. 3 shows an example of a system that supports a conscious or subconscious learning feedback loop using wearable-based tactile indications in accordance with aspects of the present disclosure.

FIG. 4 shows an example of a system that supports a conscious or subconscious learning feedback loop using wearable-based tactile indications in accordance with aspects of the present disclosure.

FIG. 5 shows an example of a graphical user interface (GUI) that supports a conscious or subconscious learning feedback loop using wearable-based tactile indications in accordance with aspects of the present disclosure.

FIG. 6 shows a block diagram of an apparatus that supports a conscious or subconscious learning feedback loop using wearable-based tactile indications in accordance with aspects of the present disclosure.

FIG. 7 shows a block diagram of a wearable application that supports a conscious or subconscious learning feedback loop using wearable-based tactile indications in accordance with aspects of the present disclosure.

FIG. 8 shows a diagram of a system including a device that supports a conscious or subconscious learning feedback loop using wearable-based tactile indications in accordance with aspects of the present disclosure.

FIGS. 9 and 10 show flowcharts illustrating methods that support feedback loop using wearable-based tactile indications in accordance with aspects of the present disclosure.

### DETAILED DESCRIPTION

Some wearable devices are configured to collect physiological data from users, and provide feedback regarding the acquired physiological data. In some cases, feedback provided by wearable devices is in the form of messages and alerts displayed on a graphical user interface (GUI) of the wearable device or a related user device. However, in order for such feedback messages to be useful, a user may be required to pause what they are doing to view and engage with the feedback, thereby decreasing the probability that the user will actually view and act upon the provided feedback. Moreover, providing feedback via GUIs is counter-productive to people's desire to limit or reduce screen time.

Some other wearable devices may be able to provide feedback to users without GUIs. For example, some wearables may enable users to trigger guided meditation sessions where the wearable device provides tactile or audio feedback to the user to help them relax. However, such guided meditation sessions (and similar feedback mechanisms) still require the user to pause what they are doing, and consciously/actively initiate the guided meditation session. Accordingly, these guided meditation sessions may be viewed as a burden to some users, thereby reducing the likelihood that users participate in guided meditation sessions and benefit from the feedback provided in the sessions.

Accordingly, aspects of the present disclosure are directed to techniques implemented by wearable devices that are configured to provide users with feedback in order to teach or guide the users to help them achieve some goal or learning objective. Such feedback techniques may be used to consciously or subconsciously teach users about their own bodies and/or their surrounding environments, and slowly train the users to learn or take actions to help them live healthier lives, manage stress more effectively, and achieve other objectives. Stated differently, aspects of the present disclosure are directed to feedback techniques implemented by wearable devices that may serve as a “sixth sense” to help train the user to achieve some goal or learning objective.

For example, a wearable device may identify a trigger event for providing feedback to the user. The satisfaction of the trigger event may be based on physiological data collected from the user via the wearable device, based on data associated with the user's surroundings (e.g., weather data, air quality data, etc.), based on data collected from another application (e.g., a navigational application, a calendar application, etc.), or any combination thereof. For instance, a trigger event may be identified if the user is experiencing stress, if the user has high blood pressure, if the user's surroundings exhibit poor air quality, and the like. Subsequently, the wearable device may provide feedback indications (e.g., vibrations, audio tones, changing temperature or pressure of the wearable device) to the user in accordance with a feedback loop to help the user (consciously or subconsciously) achieve some learning objective or goal associated with the trigger event. As such, the feedback indications may act as a “sixth sense” to help the user learn and realize when they are stressed and may need to calm down, or help the user learn when their blood pressure is rising to help them take actions to reduce their blood pressure.

After providing the feedback indications, the wearable device may monitor the user's physiological response and modify the feedback loop based on the user's physiological response to the feedback indications. In particular, the feedback loop may be adjusted based on whether or not the provided feedback was helpful in leading the user toward their learning objective(s). For instance, if the user's blood pressure did not decrease following the feedback, the wearable device may increase the magnitude of subsequent tactile vibrations to more forcefully indicate the user should take actions to reduce their blood pressure. Conversely, if the user's blood pressure did lower following the feedback indications, the wearable device may gently reduce the magnitude of subsequent tactile vibrations in order to taper off the feedback provided to the user, and help the user identify (on their own) when they need to take actions to reduce their blood pressure.

In general, the feedback loops described herein may help the user learn or achieve a wide array of goals or learning objectives, such as lowering their heart rate, lowering their stress levels, adjusting their blood pressure and/or blood sugar content, controlling their running or walking cadence (e.g., matching their running/walking cadence with their heart rate), and the like. The feedback loops described herein may also help the user achieve other learning objectives, such as learning or adapting to the Earth's magnetic field, weather conditions (e.g., air pressure, air pollution, allergens), hyperspectrum, help the user learn to navigate to navigational destinations, and the like. Furthermore, feedback loops described herein may facilitate shared sensing, or help users learn or adapt to what others are feeling. For example, feedback loops may be used to help users realize when their spouse or family member is experiencing stress, help a teacher learn when their students are becoming disengaged, help a yoga instructor learn to adapt their classes to help students relax, and the like.

Aspects of the disclosure are initially described in the context of systems supporting physiological data collection from users via wearable devices. Additional aspects of the disclosure are described in the context of an example GUI. Aspects of the disclosure are further illustrated by and described with reference to apparatus diagrams, system diagrams, and flowcharts that relate to feedback loop using wearable-based tactile indications.

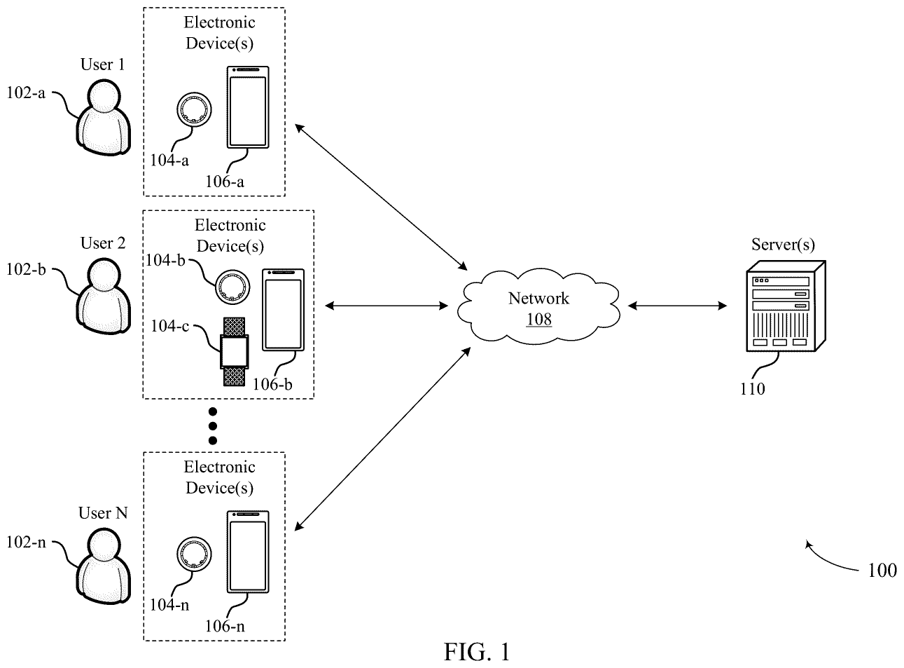

FIG. 1 illustrates an example of a system 100 that supports a conscious or subconscious learning feedback loop using wearable-based tactile indications in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the car, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled with one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled with the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled with one or more servers 110 via wired or wireless communication protocols. For example, as shown in FIG. 1, the electronic devices (e.g., user devices 106) may be communicatively coupled with one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled with the user device 106-a, where the user device 106-a is communicatively coupled with the servers 110 via the network 108. In additional or alternative cases, wearable devices 104 (e.g., rings 104, watches 104) may be directly communicatively coupled with the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage. In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for providing users with tactile or audio feedback in accordance with a learning feedback loop in order to help the user 102 learn or achieve some learning objective. For example, a wearable device 104 of the system 100 may acquire physiological data from a user 102, and identify a satisfaction of a trigger event for providing feedback to the user 102 based on the physiological data. For instance, a trigger event may be identified if the user 102 is experiencing stress, if the user 102 has high blood pressure, and the like. Subsequently, the wearable device 104 may provide feedback indications (e.g., vibrations, audio tones, changing temperature or pressure of the wearable device 104) to the user 102 in accordance with a feedback loop to help the user 102 (consciously or subconsciously) achieve some learning objective or goal associated with the trigger event.

As such, the feedback indications may act as a “sixth sense” to help the user 102 learn and realize when they are stressed and may need to calm down, or help the user 102 learn when their blood pressure is rising to help them take actions to reduce their blood pressure. In other words, aspects of the present disclosure are directed to feedback techniques that provide users with gentle “nudges” to help the users consciously or subconsciously learn or achieve certain learning objectives.

After providing the feedback indications, the wearable device 104 may monitor the user's physiological response and modify the feedback loop based on the user's physiological response to the feedback indications. For instance, if the user's blood pressure did not decrease following the feedback, the wearable device 104 may increase the magnitude of subsequent tactile vibrations to more forcefully indicate the user 102 should perform actions to reduce their blood pressure. Conversely, if the user's blood pressure did lower following the feedback indications, the wearable device 104 may gently reduce the magnitude of subsequent tactile vibrations in order to taper off the feedback provided to the user 102, and help the user 102 identify (on their own) when they need to take actions to reduce their blood pressure.

Techniques described herein may help users 102 learn or achieve (either consciously or subconsciously) one or more learning objectives by providing feedback to the users 102 in accordance with a learning feedback loop. In this regard, aspects of the present disclosure may be implemented by wearable devices 104 so that the wearable devices 104 can serve as a “sixth sense” to help the users 102 adapt to their own physiological condition, and/or their surrounding conditions.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

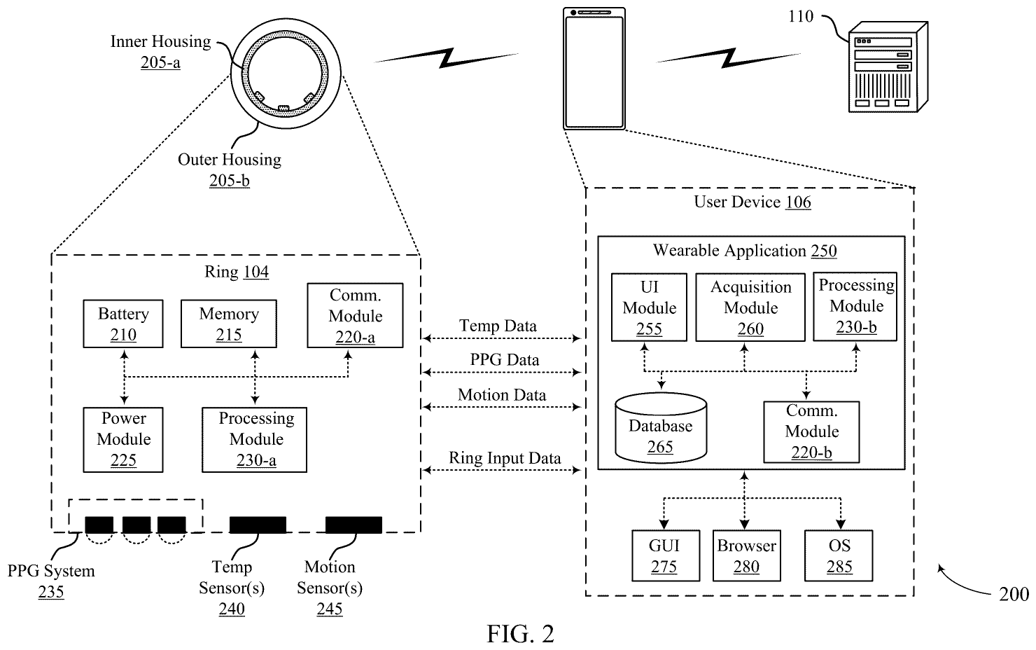

FIG. 2 illustrates an example of a system 200 that supports a conscious or subconscious learning feedback loop using wearable-based tactile indications in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled with one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled with the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the quantity of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The quantity and ratio of transmitters and receivers included in the PPG system 235 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a quantity of acceleration peaks within one or more periods of time (e.g., one or more 30 second to 1 minute periods). Intensity values may indicate a quantity of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the quantity of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the quantity of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that may use relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Duc to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the quantity of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

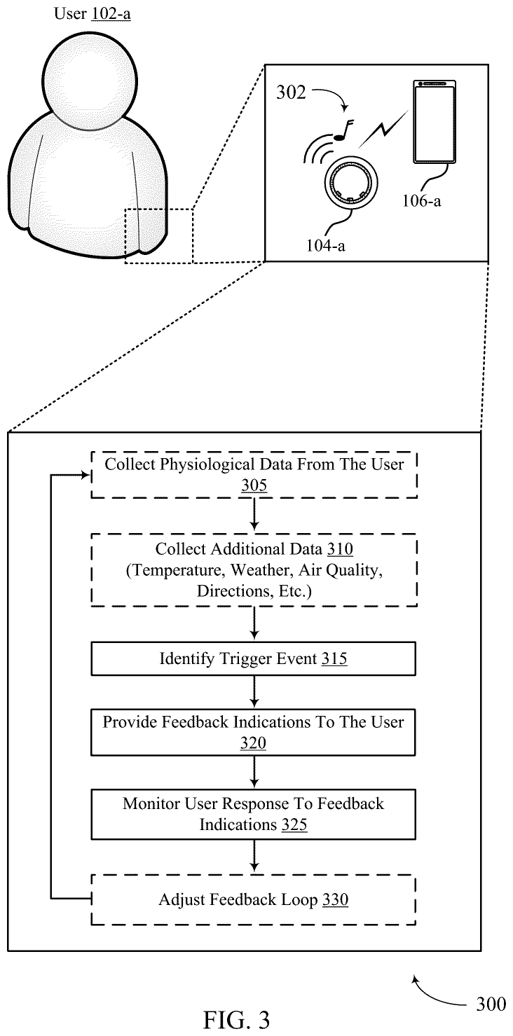

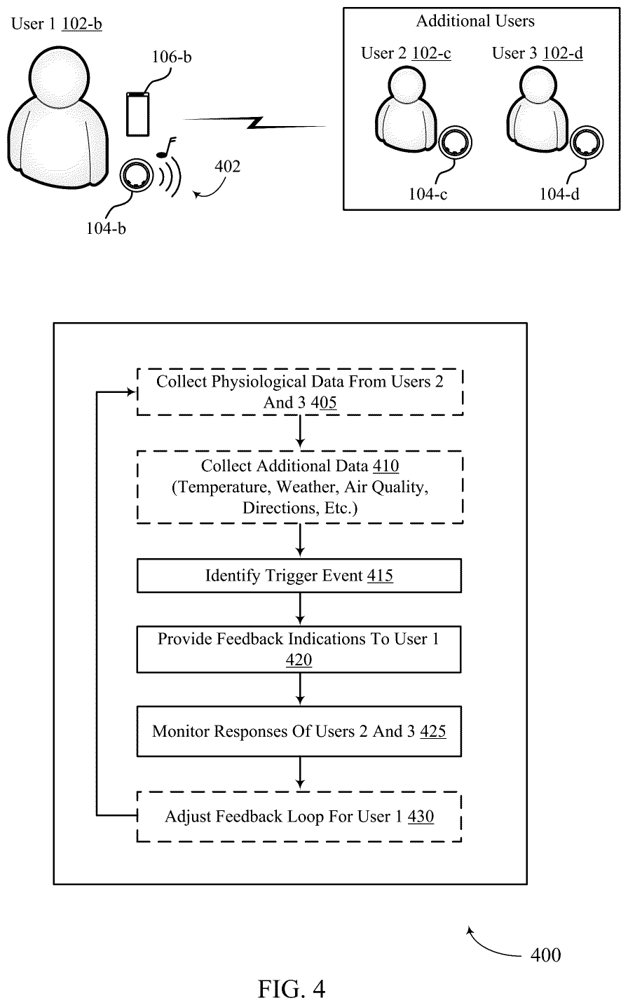

In some implementations, the various devices of the system 200 may support techniques for providing users 102 with tactile or audio feedback in accordance with a learning feedback loop in order to help the user 102 learn or achieve some learning objective. Attendant advantages of the feedback techniques described herein may be further shown and described with reference to FIGS. 3 and 4.

FIG. 3 shows an example of a system 300 that supports a conscious or subconscious learning feedback loop using wearable-based tactile indications in accordance with aspects of the present disclosure. Aspects of the system 300 may implement, or be implemented by, aspects of the system 100, the system 200, or both. In particular, the system 300 shown in FIG. 3 includes a user 102-a, a wearable device 104-a, and a user device 106-a, which may be examples of corresponding users 102 and devices described herein.

As described previously herein, the wearable device 104-a (e.g., wearable ring device, wrist-worn wearable, wearable strap, etc.) may be configured to acquire physiological data from the user 102-a. The wearable device 104-a may be configured to process physiological data from the user 102-a, and/or communicate acquired physiological data to the user device 106-a for processing (e.g., so that the user device 106-a and/or servers 110 may process the physiological data).

In some aspects, the wearable device 104-a and/or the user device 106-a may be configured to provide tactile or audible feedback (e.g., feedback 302) to the user 102-a in accordance with a learning feedback loop in order to help the user 102-a learn or perform one or more learning objectives.

For example, at 305, the wearable device 104-a may be configured to acquire physiological data from the user 102-a, such as heart rate data, HRV data, temperature data, blood oxygen data, glucose data, motion data (e.g., activity data), and the like.

In some cases, at 310, the wearable device 104-a, the user device 106-a, or both, may acquire additional data from other sources. For example, the user device 106-a may retrieve data from other applications executable on the user device 106-a, such as a weather application, a calendar application, a navigational application, and the like. In other cases, the additional information may be collected by external devices, such as a charger of the wearable device 104, smart sensors (e.g., smart thermostat), a smart Home Assistant Device (e.g., Amazon Alexa device, Google Nest device), etc. In this regard, the additional information acquired at 310 may include information associated with the environmental surroundings of the user 102-a, such as the surrounding temperature, air pressure, air quality, and the like. By way of another example, the additional information may include information associated with a navigational destination of the user 102-a, information associated with the Earth's magnetic field (e.g., North, South, East, West directions), and the like.

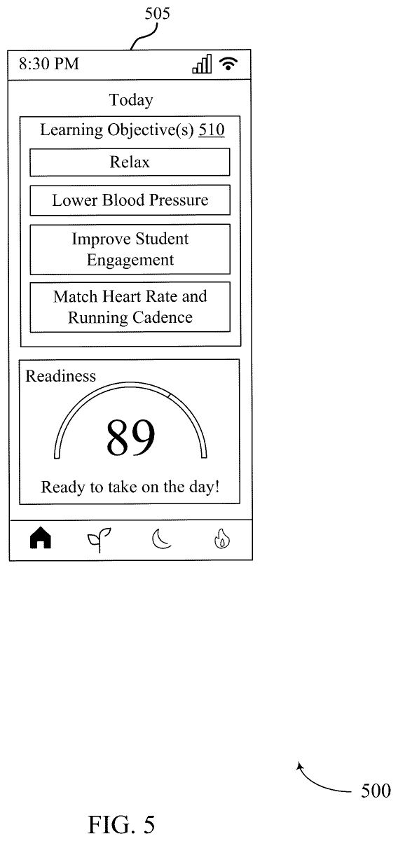

At 315, the user device 106-a, the wearable device 104-a, or both, may identify a satisfaction of a trigger event for providing tactile or audible feedback 302 to the user 102-a. The trigger event may be associated with a learning feedback loop for the user 102-a, where the learning feedback loop is configured to help the user 102-a learn or perform one or more learning objectives. In this regard, the identification of the trigger event/condition may be based on one or more learning objectives that have been selected or activated for the user 102-a. In some cases, system 300 may manually select or activate learning objectives for the user 102-a. In additional or alternative cases, the user 102-a may manually input or select learning objectives that the user 102-a wants to learn or perform. For example, as will be further shown and described with reference to FIG. 5, the user 102-a may input a desire to reduce their stress levels, to reduce their blood pressure, to adapt to poor air quality conditions, to learn which direction is North, and the like.

In this regard, the satisfaction of the trigger event may be based on what learning objectives have been activated or selected for the user 102-a. Additionally, the satisfaction of the trigger event may be based on the physiological data acquired from the user 102-a via the wearable device 104-a at 305, based on the additional data acquired at 310, or both. For example, in cases where the system 300 is configured to help the user 102-a lower their blood pressure, the system 300 (e.g., user device 106-a, wearable device 104-a, servers 110) may identify a satisfaction of the trigger condition for providing feedback 302 to the user 102-a if the user's 102-a blood pressure exceeds some threshold level.

By way of another example, in cases where the system 300 is configured to help the user 102-a reduce stress, the system 300 may identify a satisfaction of the trigger event if at least some physiological parameters associated with stress (e.g., HRV, blood pressure, heart rate, respiratory rate) satisfy one or more respective thresholds (e.g., based on a stress level metric determined using the respective physiological parameters satisfying some threshold stress level). Conversely, in other cases, the system 300 may recognize when the user 102-a is relaxed or otherwise experiencing restorative time, and may provide feedback 302 as encouragement/affirmation that the user 102-a is relaxing (e.g., provide a “cat purr” when the user 102-a is experiencing restorative time, or is otherwise relaxing). By way of another example, in cases where the user 102-a has selected a learning objective to learn navigational directions or learn the Earth's magnetic field, the system 300 may identify a satisfaction of the trigger condition when the additional data acquired at 310 indicates that the user 102-a is facing North. By way of another example, the learning objective may help the user recognize when the user is experiencing some sort of arrhythmia or other cardiac irregularity.

In other examples, the trigger condition for providing feedback 302 may be satisfied when one or more physiological parameters satisfy respective thresholds, when parameters or characteristics of the user's 102-a surrounding environment meet respective thresholds (e.g., when the temperature is above/below some temperature threshold, when the surrounding air quality is below some threshold, etc.), or any combination thereof.

At 320, the wearable device 104-a (and, in some cases, the user device 106-a) may provide tactile or audible feedback 302 to the user 102-a based on the satisfaction of the trigger event/condition. In cases where the wearable device 104-a identifies the satisfaction of the trigger event at 315, the wearable device 104-a may be configured to provide the feedback at 320 without any explicit signaling from the wearable device 106-a. Conversely, in cases where the user device 106-a processes the data and identifies the satisfaction of the trigger event, the wearable device 106-a may transit one or more feedback instructions to the wearable device 104-a, where the feedback instructions indicate for the wearable device 104-a to provide the tactile or audible feedback 302 in accordance with the learning feedback loop.

The feedback 302 provided by the wearable device 104-a may include, but is not limited to, tactile vibrations, audio sounds, and the like. In other cases, by the wearable device 104-a may provide tactile feedback 302 by changing a temperature of the cover or some component of the wearable device, adjusting a pressure exerted by the wearable device 104-a on the tissue of the user 102-a, and the like. In additional or alternative cases, the feedback 302 may include visible feedback 302 (e.g., changing a color of LEDs of the wearable device 104-a, strobing or flashing LEDs using some pattern, etc.).

The feedback 302 may be provided to the user 102-a at 320 consciously, subconsciously, or both. For the purposes of the present disclosure, feedback 302 may be said to be provided to the user “consciously” if the user 102-a actively recognizes if and when the feedback 302 is being provided (e.g., feedback 302 is provided consciously if the user 102-a is aware of the feedback 302). Conversely, for the purposes of the present disclosure, feedback 302 may be said to be provided to the user “subconsciously” if the user 102-a does not actively recognize if and/or when the feedback 302 is being provided (e.g., feedback 302 is provided subconsciously if the user 102-a is not aware of the feedback 302).

In some implementations, aspects of the present disclosure may be configured to provide feedback 302 to the user 102-a subconsciously such that the feedback 302 may help the user learn or achieve the learning objectives even when the user 102-a is not actually focusing on the learning objectives, or even when the user is not aware that the user is being trained to learn or achieve the learning objectives. Thus, subconscious feedback 302 described herein may serve as a “sixth sense” to help the user achieve the respective learning objectives.

In some cases, one or more parameters or characteristics (e.g., magnitude, volume, cadence) of the feedback 302 may be selected or adjusted in order to change whether the feedback 302 is provided consciously or subconsciously. For example, tactile feedback 302 (e.g., tactile vibrations) that are provided with a magnitude below some threshold level may not be actively recognized by the user 102-a, and may therefore be provided in a subconscious manner. By way of another example, in some cases, the feedback 302 may be provided at some cadence (e.g., regularly, constantly, and/or consistently based on the satisfaction of the trigger events) so that the user 102-a does not necessarily notice if/when the user 102-a is being “nudged” via the feedback, and thus the feedback would be provided subconsciously, or would become subconscious to the user 102-a over time. Further, in some cases, as the system 300 modifies parameters or characteristics of the learning feedback loop (e.g., as the system 300 increases or decreases the magnitude of tactile vibrations), the manner in which the feedback 302 is provided to the user may change from conscious feedback to subconscious feedback, or vice versa, depending on the parameters/characteristics of the learning feedback loop and/or the feedback 302 itself.

At 325, the wearable device 104-a may monitor the user's 102-a response to the feedback 302 provided at 320. In other words, the wearable device 104-a may acquire additional physiological data from the user 102-a in order to monitor how the user 102-a responds to the feedback 302 (e.g., whether or not the user 102-a adjusted their behavior, or modified their responses based on the provided feedback 302). Stated differently, the system 300 may monitor the user's 102-a response to the feedback 302 in order to evaluate whether or not the feedback 302 was successful in helping the user 102-a learn or perform the one or more learning objectives. For example, in cases where the feedback 302 was provided to help the user 102-a recognize and lower their high blood pressure, the wearable device 104-a may monitor the user's 102-a blood pressure to see if the user 102-a took actions to lower their blood pressure in response to the feedback 302.

At 330, the system 300 (e.g., wearable device 104-a, user device 106-a, servers 110, etc.) may selectively modify one or more parameters of the learning feedback loop (if necessary). The system 300 may adjust parameters of the learning feedback loop based on providing the feedback 302 to the user 102-a at 320, and based on monitoring the user's 102-a response to the feedback at 325. Parameters of the learning feedback loop that may be adjusted may include, but are not limited to, a magnitude or volume of the feedback 302 (e.g., make the feedback stronger or weaker), a threshold associated with the trigger event (e.g., trigger the feedback earlier/later, or more/less often), a feedback type (e.g., change from tactile to audible feedback, or vice versa), or any combination thereof.

In particular, if the system 300 determines (at 325) that the feedback 302 was successful in helping the user 102-a learn or perform the learning objectives, the system 300 may be configured to eliminate, reduce, or otherwise “taper back” future occurrences of the feedback 302 that may be provided in accordance with the learning feedback loop. By tapering off the feedback 302 provided by the feedback loop, techniques described herein may help users learn to recognize trigger events on their own (e.g., without explicit feedback) over time, and take actions to address the trigger events autonomously, thereby training the users 102 to lead healthier, more fulfilling lives. In this regard, as the user 102-a becomes better at recognizing certain trigger events associated with their learning objectives, the user 102-a may be able to modify their behaviors autonomously to achieve the learning objectives with less and less feedback 302, then eventually with no feedback 302.

Conversely, if the system 300 determines (at 325) that the feedback 302 was not successful in helping the user 102-a learn or perform the learning objectives, the system 300 may be configured to increase, change, or otherwise modify future occurrences of the feedback 302 that may be provided in accordance with the learning feedback loop.

For example, in cases where the learning objective is configured to help the user 102-a lower their blood pressure, the system 300 may determine that the user's blood pressure did not lower after providing the feedback 302. In this example, the system 300 may increase a magnitude of the tactile or audible feedback 302 so that future instances of the feedback 302 provided in accordance with the learning feedback loop are more forceful or noticeable. Conversely, in cases system 300 determines that the user's blood pressure did lower after providing the feedback 302, the system 300 may reduce a magnitude of the feedback 302 in order to “taper back” the feedback 302 as the user 102-a learns to recognize and reduce their high blood pressure on their own.

While the “learning objectives” of FIG. 3 are largely described in the context of learning objectives to help users recognize and adjust (e.g., lower) their stress levels and/or blood pressure, these are provided solely for illustrative purposes. In this regard, the system 300 illustrated in FIG. 3 may be implemented in the context of a wide variety of learning objectives, such as learning objectives to help the user 102-a control their running or walking cadence (e.g., matching their running/walking cadence with their heart rate), learn to keep their heart rate within defined heart rate “zones” during exercise, learn or adapt to the Earth's magnetic field, weather conditions (e.g., air pressure, air pollution, allergens), hyperspectrum, help the user 102-a learn to navigate to certain locations/navigational destinations, and the like.

For example, in cases where the learning objective is intended to help the user 102-a learn directions or learn Earth's magnetic field, the additional data collected at 310 may include navigational and/or positional data associated with a geographical location and/or orientation of the user 102-a. In some cases, the additional data may be collected via other applications executable on the user device 106-a, such as a navigational application or other GPS application. In this example, the system 300 (e.g., wearable device 104-a, user device 106-a, servers 110) may determine when the user 102-a is facing magnetic North based on the additional data collected at 310, and may provide the feedback 302 to the user 102-a when the additional data indicates that the user 102-a is facing magnetic North (or some other direction).

By way of another example, in cases where the learning objective is intended to help the user 102-a learn navigational directions (e.g., learn how to travel from Location A to Location B), the additional data collected at 310 may include navigational and/or positional data associated with a geographical location and/or orientation of the user 102-a. In some cases, the additional data may be collected via other applications executable on the user device 106-a, such as a navigational application or other GPS application. In this example, the system 300 (e.g., wearable device 104-a, user device 106-a, servers 110) may determine when the user 102-a is approaching an upcoming step or turn for traveling from their current position to some navigational destination (e.g., identify an upcoming turn, an upcoming entrance/exit ramp, etc.), and may provide the feedback 302 to alert the user 102-a of the upcoming step. In some cases, the feedback 302 may be provided using different parameters (e.g., different tactile or audible patterns, etc.) to indicate characteristics of the upcoming step in the navigational guidance (e.g., first tactile pattern for an upcoming left turn, a second tactile pattern for an upcoming right turn). In such cases, the system 300 may monitor the user's geographical location at 325 in order to see whether the user 102-a took the correct step to travel to the navigational destination. If the user 102-a correctly responded to the feedback 302 (e.g., made the correct turn, took the correct entrance/exit ramp), then the system 300 may adjust the feedback 302 of the learning feedback loop. For instance, the system 300 may reduce a magnitude or volume of future feedback 302, or remove differentiations of types of feedback 302 (e.g., so that future feedback 302 does not use different patterns for upcoming left/right turns).

It has been found that a user 102-a may increase their physical performance and/or stimulate their heart by matching movements (such as a running or walking cadence) with their heart rate. In effect, the periodic movements act as a “second heart rate” to help improve cardiac performance. As such, in another example, the system 300 may be used to help the user 102-a match their running/walking cadence to their heart rate. For example, the physiological data collected at 305 may include motion data (e.g., data used to determine a running/walking cadence) and heart rate data. In this example, the system 300 may identify the satisfaction of the trigger event at 315 if the user's running or walking cadence differs from the user's heart rate by some threshold amount. In this example, the system 300 may cause the wearable device 104-a to provide the feedback 302 at 320 to help the user 102-a match their running/walking cadence with their heart rate. As noted previously herein, the system 300 may utilize different parameters of the feedback 302 to indicate whether the user 102-a needs to increase or decrease their running/walking cadence to match their heart rate (e.g., first tactile pattern to increase cadence, a second tactile pattern to decrease cadence). In such cases, the system 300 may monitor the user's physiological data (e.g., motion data, heart rate data) at 325 in order to see whether the user 102-a correctly adjusted their cadence to match their heart rate. If the user 102-a correctly responded to the feedback 302 (e.g., made the correct adjustments to their running/walking cadence), then the system 300 may adjust the feedback 302 of the learning feedback loop. For instance, the system 300 may reduce a magnitude or volume of future feedback 302, or remove differentiations of types of feedback 302 (e.g., so that future feedback 302 does not use different patterns indicating whether to increase/decrease cadence).

FIG. 4 shows an example of a system 400 that supports a conscious or subconscious learning feedback loop using wearable-based tactile indications in accordance with aspects of the present disclosure. Aspects of the system 400 may implement, or be implemented by, aspects of the system 100, the system 200, the system 300, or any combination thereof. In particular, the system 400 shown in FIG. 4 includes users 102-b, 102-c, and 102-d (e.g., Users 1, 2, and 3, respectively), wearable devices 104-b, 104-c, and 104-d, and a user device 106-b, which may be examples of corresponding users 102 and devices described herein.

In some aspects, the wearable device 104-b and/or the user device 106-b may be configured to provide tactile or audible feedback (e.g., feedback 402) to the user 102-b in accordance with a learning feedback loop in order to help the user 102-b learn or perform one or more learning objectives. In some cases, compared to the system 300 where triggers for providing tactile or audible feedback are based on the user's own physiological data, the system 400 illustrated in FIG. 4 illustrates an example where the trigger for providing tactile or audible feedback 402 is based on physiological data of other users 102 (e.g., users 102-c, 102-d). Such implementations may be useful in the context of shared sensing, to help the user 102-b determine how the other users 102-c, 102-d are feeling, and the like.

For example, at 405, the additional wearable devices 104-c, 104-d may be configured to acquire physiological data from the additional users 102-c, 102-d, such as heart rate data, HRV data, temperature data, blood oxygen data, glucose data, and the like. In some cases, the additional wearable devices 104-c, 104-d may be communicatively coupled with corresponding user devices 106 that are configured to collect the physiological data from the additional users 102-c, 102-d and transmit the physiological data to one or more servers 110 and/or the user device 106-b. In additional or alternative implementations, the system 400 may also collect physiological data from the user 102-b via the wearable device 104-b.

In some cases, at 410, the wearable device 104-b, the user device 106-b, or both, may acquire additional data from other sources. For example, the user device 106-b may retrieve data from other applications executable on the user device 106-b, such as a weather application, a calendar application, a navigational application, and the like. In other cases, the additional information may be collected by external devices, such as a charger of the wearable device 104, smart sensors, an Alexa device, etc. In this regard, the additional information acquired at 410 may include information associated with the environmental surroundings of the user 102-b and/or the additional users 102-c, 102-d, such as the surrounding temperature, air pressure, air quality, and the like.

At 415, the user device 106-b, the wearable device 104-b, or both, may identify a satisfaction of a trigger event for providing tactile or audible feedback 402 to the user 102-b. The trigger event may be associated with a learning feedback loop for the user 102-b, where the learning feedback loop is configured to help the user 102-b learn or perform one or more learning objectives. For example, in the context of FIG. 4, the learning objectives may be configured to help the user recognize the physical, mental, and/or emotional state of the additional users 102-c, 102-d.

In this regard, the identification of the trigger condition may be based on one or more learning objectives that have been selected or activated for the user 102-b. In some cases, the system may manually select or activate learning objectives for the user 102-b. In additional or alternative cases, the user 102-b may manually input or select learning objectives that the user 102-b wants to learn or perform. For example, as will be further shown and described with reference to FIG. 5, the user 102-b may input a desire to reduce their stress levels, to reduce their blood pressure, to adapt to poor air quality conditions, to learn which direction is North, and the like.

In this regard, the satisfaction of the trigger event may be based on what learning objectives have been activated or selected for the user 102-b. The satisfaction of the trigger event may be based on the physiological data acquired from the additional users 102-c, 102-d via the additional wearable devices 104-c, 104-d at 405, based on the additional data acquired at 410, or both. For example, in cases where the system is configured to help the user recognize physical/mental/emotional states of the additional users 102-c, 102-d, the system (e.g., user device 106-b, wearable device 104-b, servers 110) may identify a satisfaction of the trigger condition for providing feedback 402 to the user 102-b if the physiological data collected from the additional users 102-c, 102-d indicates that the additional users are experiencing some threshold level of stress, or are otherwise experiencing some physical, mental, or emotional state.

The “shared sensing” implementations shown and described in FIG. 4 may be implemented in a variety of different contexts, and to help the user 102-b achieve a variety of different learning objectives. For example, the system 400 may help the user 102-b determine when additional users 102-c, 102-c (e.g., spouse, friend, family member) is having a rough time, and could use a friendly check-in or words of encouragement. By way of another example, in cases where the user 102-b is a teacher and the additional users 102-c, 102-d are the teacher's students, the system 400 may help the teacher (e.g., user 102-b) recognize when his/her students are becoming disengaged, which may help the teacher adjust the lesson plan or environment to help improve engagement. Similarly, in cases where the user 102-b is a yoga instructor and the additional users 102-c, 102-d are the students, the system 400 may help the yoga instructor (e.g., user 102-b) recognize if or how relaxed the students are, which may help the teacher adjust the poses or environment of the yoga class to help improve relaxation or achieve some other goal.

At 420, the wearable device 104-b (and, in some cases, the user device 106-b) may provide tactile or audible feedback 402 to the user based on the satisfaction of the trigger condition. In cases where the wearable device 104-b identifies the satisfaction of the trigger event at 415, the wearable device 104-b may be configured to provide the feedback at 420 without any explicit signaling from the wearable device 106-b. Conversely, in cases where the user device 106-b processes the data and identifies the satisfaction of the trigger event, the wearable device 106-b may transit one or more feedback instructions to the wearable device 104-b, where the feedback instructions indicate for the wearable device 104-b to provide the tactile or audible feedback 402 in accordance with the learning feedback loop.

The feedback 402 provided by the wearable device 104-b may include, but is not limited to, tactile vibrations, audio sounds, and the like. In other cases, the wearable device 104-b may provide the feedback 402 by changing a temperature of the cover or some component of the wearable device, adjusting a pressure exerted by the wearable device 104-b on the tissue of the user 102-b, and the like. In additional or alternative cases, the feedback 402 may include visible feedback (e.g., changing a color of LEDs of the wearable device 104-b, strobing or flashing LEDs using some pattern, etc.).

At 425, the wearable device 104-b may monitor the response(s) of the additional users 102-c, 102-d after providing the feedback 402 to the first user 102-b at 420. In this regard, the feedback 302 may be provided to the first user 102-b, where the evaluation of the success of the feedback may be based on the physiological responses of the additional users 102-c, 102-d (e.g., under the assumption that the first user 102-b takes some action in response to the feedback 302 to help adjust the physiological responses of the additional users 102-c, 102-d).

In other words, the wearable device 104-b may acquire additional physiological data from the additional users 102-c, 102-d in order to monitor whether the first user 102-b took actions in response to the feedback 402 to help adjust the physiological data of the additional users 102-c, 102-d. In some cases, the system 400 may additionally monitor the response of the first user 102-b in order to evaluate whether or not the feedback was successful in helping the user 102-b learn or achieve the learning objectives.

For example, in cases where the feedback 402 was provided to help the user 102-b (e.g., teacher) improve the engagement of her students, the wearable devices 104-c, 104-d may monitor physiological data of the additional users 102-c, 102-d to determine whether the physiological states of the additional users 102-c, 102-d changed after providing the feedback 302 to the first user 102-b. In cases where the physiological data of the additional users 102-c, 102-d suggests that the engagement if the additional users 102-c, 102-d increased, the system 400 may determine that the user 102-b likely took actions in response to the feedback 302 to help improve engagement, meaning the feedback 302 was successful in helping the user 102-b achieve the learning objective (e.g., learning objective to increase engagement of the additional users 102-c, 102-d). Conversely, in cases where the physiological data of the additional users 102-c, 102-d suggests that the engagement if the additional users 102-c, 102-d did not increase following the feedback 302, the system 400 may determine that the user 102-b likely did not take any actions in response to the feedback 302 to help improve engagement (or that actions taken by the user 102-b were unsuccessful to improve engagement). As such, in this example, the system 400 may determine that the feedback 302 was unsuccessful in helping the user 102-b achieve the learning objective.

At 430, the system (e.g., wearable device 104-b, user device 106-b, servers 110, etc.) may selectively modify one or more parameters of the learning feedback loop (if necessary). The system 400 may adjust parameters of the learning feedback loop based on providing the feedback 402 to the user 102-b at 420, and based on monitoring the additional user's 102-c, 102-d response after providing the feedback 302 to the first user 102-b at 425. Parameters of the learning feedback loop that may be adjusted may include, but are not limited to, a magnitude or volume of the feedback 402 (e.g., make the feedback stronger or weaker), a threshold associated with the trigger event (e.g., trigger the feedback earlier/later, or more/less often), a feedback type (e.g., change from tactile to audible feedback, or vice versa), or any combination thereof.

In particular, if the system determines (at 425) that the feedback 402 was successful in helping the user learn or perform the learning objectives, the system 400 may be configured to eliminate, reduce, or otherwise “taper back” future occurrences of the feedback 402 that may be provided in accordance with the learning feedback loop. Conversely, if the system 400 determines (at 425) that the feedback 402 was not successful in helping the user 102-b learn or perform the learning objectives, the system 400 may be configured to increase, change, or otherwise modify future occurrences of the feedback 402 that may be provided in accordance with the learning feedback loop. In this regard, as the user 102-b becomes better at recognizing physical, mental, and/or emotional states of the additional users 102-c, 102-d in accordance with the learning objectives, the user 102-b may be able to modify their behaviors autonomously to achieve the learning objectives with less and less feedback 402, then eventually with no feedback 402.

FIG. 5 shows an example of a GUI 500 that supports a conscious or subconscious learning feedback loop using wearable-based tactile indications in accordance with aspects of the present disclosure. The GUI 500 may implement, or be implemented by, aspects of the systems 100-400, or any combination thereof. For example, the GUI 500 may be implemented for a user at a user device 106 connected to a wearable device 104 (e.g., a wearable ring device, watch, wrist-worn wearable, necklace, or any other wearable device), as described with reference to FIGS. 1-4.

The GUI 500 illustrates an application page 505 that may be displayed to a user via the GUI 500. For example, the application page 505 may be an example application page of a wearable application 250 executable on a user device 106 that may be displayed via a GUI 275 of the wearable device 106. In some examples, the user 102 may open the application page 505 to see scores belonging to the user. For example, the application page 505-a may display a Sleep Score, a Readiness Score, and the like.

In some examples, as described previously herein, a user 102 may be able to manually select or activate one or more learning objectives that the user is interested in, or which the user 102 wants to learn or achieve. For example, the application page 505 illustrates a set of candidate learning objectives 510 that the user may select, activate, or otherwise opt-into. The set of candidate learning objectives 510 may include any learning objectives described herein, such as a learning objective to help the user relax (e.g., learning objective to reduce stress), a learning objective to help the user recognize and adjust (e.g., reduce) their blood pressure, a learning objective to help a teacher improve engagement of his/her students, a learning objective to help train the user 102 to match their running/walking cadence with their heart rate, or any combination thereof.

In some cases, the system may be configured to recommend learning objectives 510 to the user 102 via the application page 505. For example, the system may evaluate physiological data (and/or additional data) associated with the user 102, and recommend one or more learning objectives 510 to the user 102 via the application page based on the analysis of the physiological data.

In this example, the user 102 may be able to select or activate one or more of the candidate learning objectives 510 such that the system (e.g., system 100, 200, 300, 400) can begin providing feedback to the user 102 in accordance with learning feedback loops associated with the respective learning objectives, as described herein.

In some implementations, the application page 505 may display information that enables the user 102 to manually adjust one or more parameters or characteristics of a learning feedback loop associated with a selected or activated learning objective 510. For example, via the application page 505 (or another application page 505), the user 102 may be able to select or adjust thresholds of trigger events used to provide feedback in accordance with the learning feedback loop (e.g., adjust when/how often feedback is provided to help the user 102 learn/achieve the learning objective 510), what type of feedback is provided (e.g., whether feedback will be tactile or audible feedback), a magnitude or volume of the feedback, and the like.

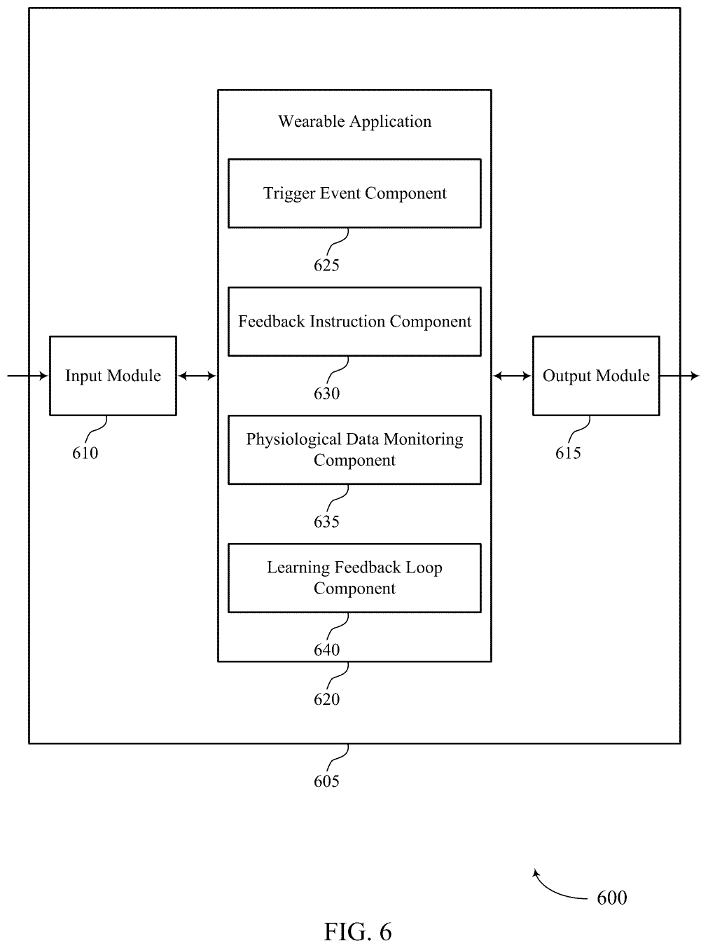

FIG. 6 shows a block diagram 600 of a device 605 that supports a conscious or subconscious learning feedback loop using wearable-based tactile indications in accordance with aspects of the present disclosure. The device 605 may include an input module 610, an output module 615, and a wearable application 620. The device 605 may also include a processor. Each of these components may be in communication with one another (e.g., via one or more buses).

The input module 610 may provide a means for receiving information such as packets, user data, control information, or any combination thereof associated with various information channels (e.g., control channels, data channels, information channels related to illness detection techniques). Information may be passed on to other components of the device 605. The input module 610 may utilize a single antenna or a set of multiple antennas.

The output module 615 may provide a means for transmitting signals generated by other components of the device 605. For example, the output module 615 may transmit information such as packets, user data, control information, or any combination thereof associated with various information channels (e.g., control channels, data channels, information channels related to illness detection techniques). In some examples, the output module 615 may be co-located with the input module 610 in a transceiver module. The output module 615 may utilize a single antenna or a set of multiple antennas.

For example, the wearable application 620 may include a trigger event component 625, a feedback instruction component 630, a physiological data monitoring component 635, a learning feedback loop component 640, or any combination thereof. In some examples, the wearable application 620, or various components thereof, may be configured to perform various operations (e.g., receiving, monitoring, transmitting) using or otherwise in cooperation with the input module 610, the output module 615, or both. For example, the wearable application 620 may receive information from the input module 610, send information to the output module 615, or be integrated in combination with the input module 610, the output module 615, or both to receive information, transmit information, or perform various other operations as described herein.

The trigger event component 625 may be configured as or otherwise support a means for identifying, at an application executing on a user device, a satisfaction of a trigger event for providing the tactile or audible feedback to a user via the wearable device. The feedback instruction component 630 may be configured as or otherwise support a means for communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event. The physiological data monitoring component 635 may be configured as or otherwise support a means for monitoring physiological data collected from the user, an additional user, or both, based at least in part on communicating the one or more feedback instructions. The learning feedback loop component 640 may be configured as or otherwise support a means for selectively modifying one or more parameters of the learning feedback loop based at least in part on an evaluation of the physiological data relative to the one or more learning objectives.

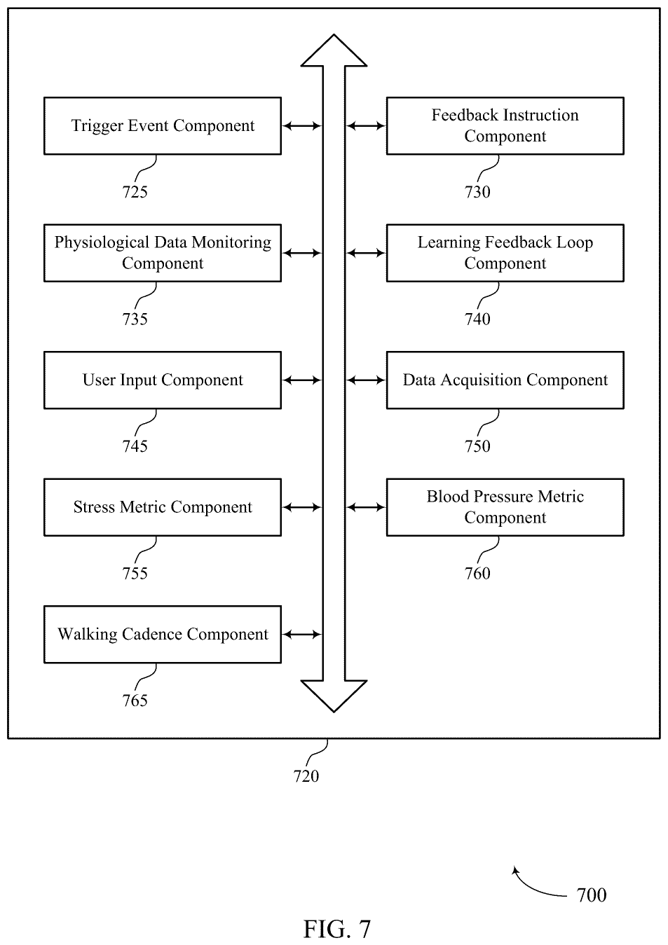

FIG. 7 shows a block diagram 700 of a wearable application 720 that supports a conscious or subconscious learning feedback loop using wearable-based tactile indications in accordance with aspects of the present disclosure. The wearable application 720 may be an example of aspects of a wearable application or a wearable application 620, or both, as described herein. The wearable application 720, or various components thereof, may be an example of means for performing various aspects of feedback loop using wearable-based tactile indications as described herein. For example, the wearable application 720 may include a trigger event component 725, a feedback instruction component 730, a physiological data monitoring component 735, a learning feedback loop component 740, a user input component 745, a data acquisition component 750, a stress metric component 755, a blood pressure metric component 760, a walking cadence component 765, or any combination thereof. Each of these components may communicate, directly or indirectly, with one another (e.g., via one or more buses).

The trigger event component 725 may be configured as or otherwise support a means for identifying, at an application executing on a user device, a satisfaction of a trigger event for providing the tactile or audible feedback to a user via the wearable device. The feedback instruction component 730 may be configured as or otherwise support a means for communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event. The physiological data monitoring component 735 may be configured as or otherwise support a means for monitoring physiological data collected from the user, an additional user, or both, based at least in part on communicating the one or more feedback instructions. The learning feedback loop component 740 may be configured as or otherwise support a means for selectively modifying one or more parameters of the learning feedback loop based at least in part on an evaluation of the physiological data relative to the one or more learning objectives.

In some examples, the user input component 745 may be configured as or otherwise support a means for receiving, via a user device associated with the user, a user input indicating the one or more learning objectives, where identifying the satisfaction of the trigger event is based at least in part on receiving the user input.

In some examples, the physiological data monitoring component 735 may be configured as or otherwise support a means for receiving, at the application executing on the user device, additional physiological data collected from the user via the wearable device, collected from an additional user via an additional wearable device, or both, where the satisfaction of the trigger event is based at least in part on the additional physiological data.

In some examples, the data acquisition component 750 may be configured as or otherwise support a means for receiving, from one or more additional applications executing on the user device, additional data associated with environmental surroundings of the user, a schedule of the user, a navigational destination of the user, or any combination thereof, where the satisfaction of the trigger event is based at least in part on the additional data.

In some examples, the physiological data monitoring component 735 may be configured as or otherwise support a means for receiving, at the application executing on the user device, additional physiological data collected from the user via the wearable device, where the additional physiological data comprises heart rate data, HRV data, temperature data, or any combination thereof. In some examples, the stress metric component 755 may be configured as or otherwise support a means for determining a stress metric associated with the user based at least in part on the heart rate data, the HRV data, temperature data, or any combination thereof, the stress metric associated with a relative level of stress or relaxation experienced by the user, where identifying the satisfaction of the trigger event is based at least in part on the stress metric satisfying a threshold stress level.

In some examples, the physiological data monitoring component 735 may be configured as or otherwise support a means for receiving, at the application executing on the user device, additional physiological data collected from the user via the wearable device. In some examples, the blood pressure metric component 760 may be configured as or otherwise support a means for determining a blood pressure metric associated with the user based at least in part on the additional physiological data, where identifying the satisfaction of the trigger event is based at least in part on the blood pressure metric being greater than or equal to a threshold blood pressure metric.

In some examples, the physiological data monitoring component 735 may be configured as or otherwise support a means for receiving, at the application executing on the user device, additional physiological data collected from the user via the wearable device, where the additional physiological data comprises heart rate data and motion data. In some examples, the walking cadence component 765 may be configured as or otherwise support a means for determining the running or walking cadence of the user based at least in part on the motion data. In some examples, the physiological data monitoring component 735 may be configured as or otherwise support a means for determining the heart rate of the user based at least in part on the heart rate data. In some examples, the trigger event component 725 may be configured as or otherwise support a means for determining that a difference between the heart rate and the running or walking cadence satisfies a threshold difference, where identifying the satisfaction of the trigger event is based at least in part on the difference satisfying the threshold difference.

In some examples, the physiological data monitoring component 735 may be configured as or otherwise support a means for receiving, at the application executing on the user device, additional physiological data collected from the additional user via an additional wearable device, where the satisfaction of the trigger event is based at least in part on the additional physiological data, and where monitoring the physiological data comprises monitoring the physiological data collected from the additional user.

In some examples, the tactile or audible feedback provided by the wearable device comprises one or more tactile vibrations, a temperature change of a surface of the wearable device, a pressure change exerted by the wearable device on a tissue of the user, or any combination thereof.

In some examples, the one or more feedback instructions are executable by the wearable device to provide the tactile or audible feedback to the user subconsciously in accordance with one or more characteristics of the tactile or audible feedback. In some examples, the one or more characteristics of the tactile or audible feedback comprise a magnitude, a volume, a cadence, or any combination thereof.

In some examples, the one or more parameters of the learning feedback loop comprise a magnitude or volume of the tactile or audible feedback, a threshold associated with the trigger event, a feedback type of the learning feedback loop, or any combination thereof.

In some examples, the physiological data is collected from the user via the wearable device. In some examples, the one or more learning objectives comprise an objective to reduce or manage stress, an objective to recognize or lower blood pressure, an objective to match a running or walking cadence to a heart rate, an objective to improve engagement or relaxation for other users, or any combination thereof.

In some examples, the wearable device comprises a wearable ring device.

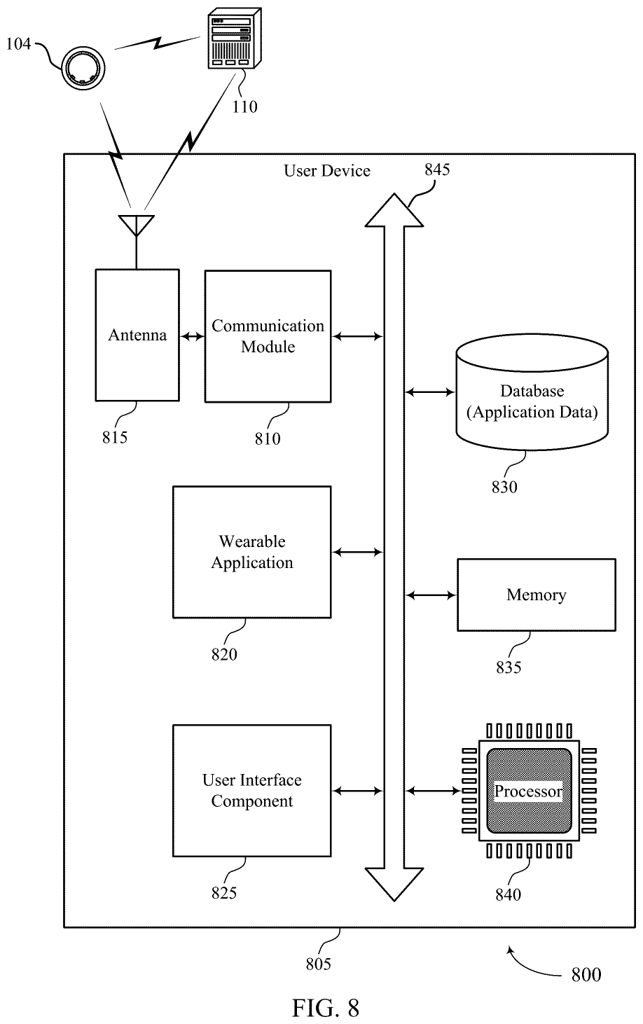

FIG. 8 shows a diagram of a system 800 including a device 805 that supports a conscious or subconscious learning feedback loop using wearable-based tactile indications in accordance with aspects of the present disclosure. The device 805 may be an example of or include the components of a device 605 as described herein.

The device 805 may include an example of a user device 106, as described previously herein. The device 805 may include components for bi-directional communications including components for transmitting and receiving communications with a wearable device 104 and a server 110, such as a wearable application 820, a communication module 810, an antenna 815, a user interface component 825, a database (application data) 830, a memory 835, and a processor 840. These components may be in electronic communication or otherwise coupled (e.g., operatively, communicatively, functionally, electronically, electrically) via one or more buses (e.g., a bus 845).

The communication module 810 may manage input and output signals for the device 805 via the antenna 815. The communication module 810 may include an example of the communication module 220-b of the user device 106 shown and described in FIG. 2. In this regard, the communication module 810 may manage communications with the ring 104 and the server 110, as illustrated in FIG. 2. The communication module 810 may also manage peripherals not integrated into the device 805. In some cases, the communication module 810 may represent a physical connection or port to an external peripheral. In some cases, the communication module 810 may utilize an operating system such as iOS®, ANDROID®, MS-DOS®, MS-WINDOWS®, OS/2®, UNIX®, LINUX®, or another known operating system. In other cases, the communication module 810 may represent or interact with a wearable device (e.g., ring 104), modem, a keyboard, a mouse, a touchscreen, or a similar device. In some cases, the communication module 810 may be implemented as part of the processor 840. In some examples, a user may interact with the device 805 via the communication module 810, user interface component 825, or via hardware components controlled by the communication module 810.

In some cases, the device 805 may include a single antenna 815. However, in some other cases, the device 805 may have more than one antenna 815, which may be capable of concurrently transmitting or receiving multiple wireless transmissions. The communication module 810 may communicate bi-directionally, via the one or more antennas 815, wired, or wireless links as described herein. For example, the communication module 810 may represent a wireless transceiver and may communicate bi-directionally with another wireless transceiver. The communication module 810 may also include a modem to modulate the packets, to provide the modulated packets to one or more antennas 815 for transmission, and to demodulate packets received from the one or more antennas 815.

The user interface component 825 may manage data storage and processing in a database 830. In some cases, a user may interact with the user interface component 825. In other cases, the user interface component 825 may operate automatically without user interaction. The database 830 may be an example of a single database, a distributed database, multiple distributed databases, a data store, a data lake, or an emergency backup database.

The memory 835 may include RAM and ROM. The memory 835 may store computer-readable, computer-executable software including instructions that, when executed, cause the processor 840 to perform various functions described herein. In some cases, the memory 835 may contain, among other things, a BIOS which may control basic hardware or software operation such as the interaction with peripheral components or devices.

The processor 840 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 840 may be configured to operate a memory array using a memory controller. In other cases, a memory controller may be integrated into the processor 840. The processor 840 may be configured to execute computer-readable instructions stored in a memory 835 to perform various functions (e.g., functions or tasks supporting a method and system for sleep staging algorithms).

For example, the wearable application 820 may be configured as or otherwise support a means for identifying, at an application executing on a user device, a satisfaction of a trigger event for providing the tactile or audible feedback to a user via the wearable device. The wearable application 820 may be configured as or otherwise support a means for communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event. The wearable application 820 may be configured as or otherwise support a means for monitoring physiological data collected from the user, an additional user, or both, based at least in part on communicating the one or more feedback instructions. The wearable application 820 may be configured as or otherwise support a means for selectively modifying one or more parameters of the learning feedback loop based at least in part on an evaluation of the physiological data relative to the one or more learning objectives.

The wearable application 820 may include an application (e.g., “app”), program, software, or other component which is configured to facilitate communications with a ring 104, server 110, other user devices 106, and the like. For example, the wearable application 820 may include an application executable on a user device 106 which is configured to receive data (e.g., physiological data) from a ring 104, perform processing operations on the received data, transmit and receive data with the servers 110, and cause presentation of data to a user 102.

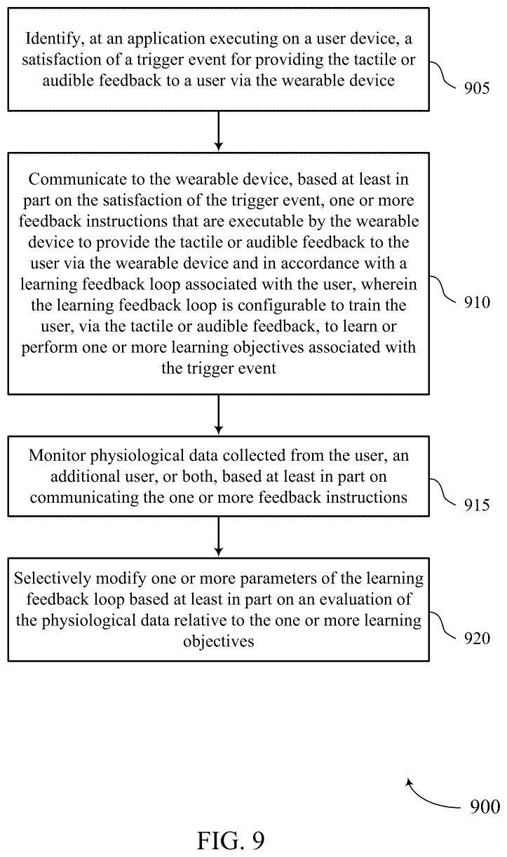

FIG. 9 shows a flowchart illustrating a method 900 that supports a conscious or subconscious learning feedback loop using wearable-based tactile indications in accordance with aspects of the present disclosure. The operations of the method 900 may be implemented by a user device or its components as described herein. For example, the operations of the method 900 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 905, the method may include identifying, at an application executing on a user device, a satisfaction of a trigger event for providing the tactile or audible feedback to a user via the wearable device. The operations of block 905 may be performed in accordance with examples as disclosed herein. In some examples, aspects of the operations of 905 may be performed by a trigger event component 725 as described with reference to FIG. 7.

At 910, the method may include communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event. The operations of block 910 may be performed in accordance with examples as disclosed herein. In some examples, aspects of the operations of 910 may be performed by a feedback instruction component 730 as described with reference to FIG. 7.

At 915, the method may include monitoring physiological data collected from the user, an additional user, or both, based at least in part on communicating the one or more feedback instructions. The operations of block 915 may be performed in accordance with examples as disclosed herein. In some examples, aspects of the operations of 915 may be performed by a physiological data monitoring component 735 as described with reference to FIG. 7.

At 920, the method may include selectively modifying one or more parameters of the learning feedback loop based at least in part on an evaluation of the physiological data relative to the one or more learning objectives. The operations of block 920 may be performed in accordance with examples as disclosed herein. In some examples, aspects of the operations of 920 may be performed by a learning feedback loop component 740 as described with reference to FIG. 7.

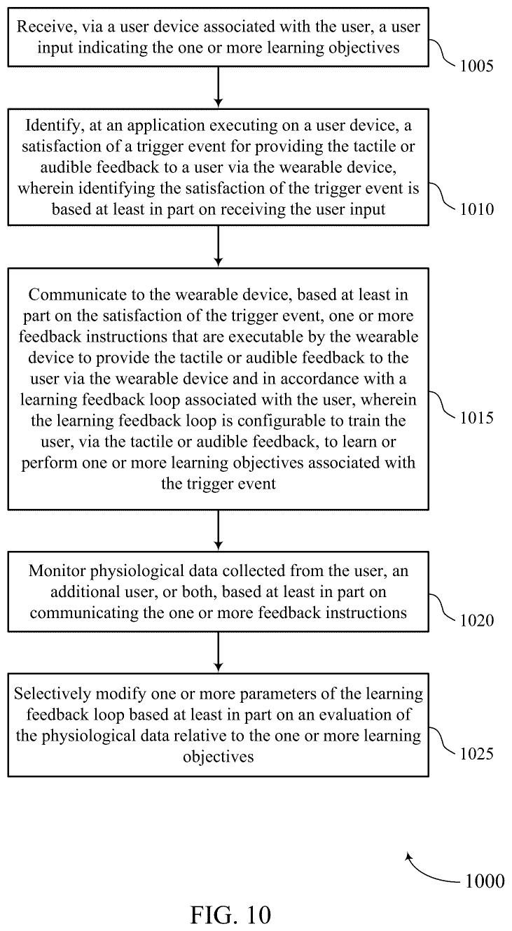

FIG. 10 shows a flowchart illustrating a method 1000 that supports a conscious or subconscious learning feedback loop using wearable-based tactile indications in accordance with aspects of the present disclosure. The operations of the method 1000 may be implemented by a user device or its components as described herein. For example, the operations of the method 1000 may be performed by a user device as described with reference to FIGS. 1 through 8. In some examples, a user device may execute a set of instructions to control the functional elements of the user device to perform the described functions. Additionally, or alternatively, the user device may perform aspects of the described functions using special-purpose hardware.

At 1005, the method may include receiving, via a user device associated with the user, a user input indicating the one or more learning objectives. The operations of block 1005 may be performed in accordance with examples as disclosed herein. In some examples, aspects of the operations of 1005 may be performed by a user input component 745 as described with reference to FIG. 7.

At 1010, the method may include identifying, at an application executing on a user device, a satisfaction of a trigger event for providing the tactile or audible feedback to a user via the wearable device, where identifying the satisfaction of the trigger event is based at least in part on receiving the user input. The operations of block 1010 may be performed in accordance with examples as disclosed herein. In some examples, aspects of the operations of 1010 may be performed by a trigger event component 725 as described with reference to FIG. 7.

At 1015, the method may include communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event. The operations of block 1015 may be performed in accordance with examples as disclosed herein. In some examples, aspects of the operations of 1015 may be performed by a feedback instruction component 730 as described with reference to FIG. 7.

At 1020, the method may include monitoring physiological data collected from the user, an additional user, or both, based at least in part on communicating the one or more feedback instructions. The operations of block 1020 may be performed in accordance with examples as disclosed herein. In some examples, aspects of the operations of 1020 may be performed by a physiological data monitoring component 735 as described with reference to FIG. 7.

At 1025, the method may include selectively modifying one or more parameters of the learning feedback loop based at least in part on an evaluation of the physiological data relative to the one or more learning objectives. The operations of block 1025 may be performed in accordance with examples as disclosed herein. In some examples, aspects of the operations of 1025 may be performed by a learning feedback loop component 740 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method is described. The method may include identifying, at an application executing on a user device, a satisfaction of a trigger event for providing the tactile or audible feedback to a user via the wearable device, communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event, monitoring physiological data collected from the user, an additional user, or both, based at least in part on communicating the one or more feedback instructions, and selectively modifying one or more parameters of the learning feedback loop based at least in part on an evaluation of the physiological data relative to the one or more learning objectives.

An apparatus is described. The apparatus may include a processor, memory coupled with the processor, and instructions stored in the memory. The instructions may be executable by the processor to cause the apparatus to identify, at an application executing on a user device, a satisfaction of a trigger event for providing the tactile or audible feedback to a user via the wearable device, communicate to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event, monitor physiological data collected from the user, an additional user, or both, based at least in part on communicating the one or more feedback instructions, and selectively modify one or more parameters of the learning feedback loop based at least in part on an evaluation of the physiological data relative to the one or more learning objectives.

Another apparatus is described. The apparatus may include means for identifying, at an application executing on a user device, a satisfaction of a trigger event for providing the tactile or audible feedback to a user via the wearable device, means for communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event, means for monitoring physiological data collected from the user, an additional user, or both, based at least in part on communicating the one or more feedback instructions, and means for selectively modifying one or more parameters of the learning feedback loop based at least in part on an evaluation of the physiological data relative to the one or more learning objectives.

A non-transitory computer-readable medium storing code is described. The code may include instructions executable by a processor to identify, at an application executing on a user device, a satisfaction of a trigger event for providing the tactile or audible feedback to a user via the wearable device, communicate to the wearable device, based at least in part on the satisfaction of the trigger event, one or more feedback instructions that are executable by the wearable device to provide the tactile or audible feedback to the user via the wearable device and in accordance with a learning feedback loop associated with the user, where the learning feedback loop is configurable to train the user, via the tactile or audible feedback, to learn or perform one or more learning objectives associated with the trigger event, monitor physiological data collected from the user, an additional user, or both, based at least in part on communicating the one or more feedback instructions, and selectively modify one or more parameters of the learning feedback loop based at least in part on an evaluation of the physiological data relative to the one or more learning objectives.

Some examples of the method, apparatuses, and non-transitory computer-readable medium described herein may further include operations, features, means, or instructions for receiving, via a user device associated with the user, a user input indicating the one or more learning objectives, where identifying the satisfaction of the trigger event may be based at least in part on receiving the user input.

Some examples of the method, apparatuses, and non-transitory computer-readable medium described herein may further include operations, features, means, or instructions for receiving, at the application executing on the user device, additional physiological data collected from the user via the wearable device, collected from an additional user via an additional wearable device, or both, where the satisfaction of the trigger event may be based at least in part on the additional physiological data.

Some examples of the method, apparatuses, and non-transitory computer-readable medium described herein may further include operations, features, means, or instructions for receiving, from one or more additional applications executing on the user device, additional data associated with environmental surroundings of the user, a schedule of the user, a navigational destination of the user, or any combination thereof, where the satisfaction of the trigger event may be based at least in part on the additional data.

Some examples of the method, apparatuses, and non-transitory computer-readable medium described herein may further include operations, features, means, or instructions for receiving, at the application executing on the user device, additional physiological data collected from the user via the wearable device, where the additional physiological data comprises heart rate data, HRV data, temperature data, or any combination thereof and determining a stress metric associated with the user based at least in part on the heart rate data, the HRV data, temperature data, or any combination thereof, the stress metric associated with a relative level of stress or relaxation experienced by the user, where identifying the satisfaction of the trigger event may be based at least in part on the stress metric satisfying a threshold stress level.

Some examples of the method, apparatuses, and non-transitory computer-readable medium described herein may further include operations, features, means, or instructions for receiving, at the application executing on the user device, additional physiological data collected from the user via the wearable device and determining a blood pressure metric associated with the user based at least in part on the additional physiological data, where identifying the satisfaction of the trigger event may be based at least in part on the blood pressure metric being greater than or equal to a threshold blood pressure metric.

Some examples of the method, apparatuses, and non-transitory computer-readable medium described herein may further include operations, features, means, or instructions for receiving, at the application executing on the user device, additional physiological data collected from the user via the wearable device, where the additional physiological data comprises heart rate data and motion data, determining the running or walking cadence of the user based at least in part on the motion data, determining the heart rate of the user based at least in part on the heart rate data, and determining that a difference between the heart rate and the running or walking cadence satisfies a threshold difference, where identifying the satisfaction of the trigger event may be based at least in part on the difference satisfying the threshold difference.

Some examples of the method, apparatuses, and non-transitory computer-readable medium described herein may further include operations, features, means, or instructions for receiving, at the application executing on the user device, additional physiological data collected from the additional user via an additional wearable device, where the satisfaction of the trigger event may be based at least in part on the additional physiological data, and where monitoring the physiological data comprises monitoring the physiological data collected from the additional user.

In some examples of the method, apparatuses, and non-transitory computer-readable medium described herein, the tactile or audible feedback provided by the wearable device comprises one or more tactile vibrations, a temperature change of a surface of the wearable device, a pressure change exerted by the wearable device on a tissue of the user, or any combination thereof.

In some examples of the method, apparatuses, and non-transitory computer-readable medium described herein, the one or more feedback instructions may be executable by the wearable device to provide the tactile or audible feedback to the user subconsciously in accordance with one or more characteristics of the tactile or audible feedback and the one or more characteristics of the tactile or audible feedback comprise a magnitude, a volume, a cadence, or any combination thereof.

In some examples of the method, apparatuses, and non-transitory computer-readable medium described herein, the one or more parameters of the learning feedback loop comprise a magnitude or volume of the tactile or audible feedback, a threshold associated with the trigger event, a feedback type of the learning feedback loop, or any combination thereof.

In some examples of the method, apparatuses, and non-transitory computer-readable medium described herein, the physiological data may be collected from the user via the wearable device and the one or more learning objectives comprise an objective to reduce or manage stress, an objective to recognize or lower blood pressure, an objective to match a running or walking cadence to a heart rate, an objective to improve engagement or relaxation for other users, or any combination thereof.

In some examples of the method, apparatuses, and non-transitory computer-readable medium described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for administering tactile or audible feedback via a wearable device, comprising:
identifying, at an application executing on a user device, a satisfaction of a trigger event for providing the tactile or audible feedback to a user via the wearable device;
communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, wherein the learning feedback loop is configurable to train the user, via the tactile or audible feedback, to learn or perform one or more learning objectives associated with the trigger event;
monitoring physiological data collected from the user, an additional user, or both, based at least in part on communicating the one or more feedback instructions; and
selectively modifying one or more parameters of the learning feedback loop based at least in part on an evaluation of the physiological data relative to the one or more learning objectives.

2. The wearable device of claim 1, further comprising:
receiving, via a user device associated with the user, a user input indicating the one or more learning objectives, wherein identifying the satisfaction of the trigger event is based at least in part on receiving the user input.

3. The wearable device of claim 1, further comprising:
receiving, at the application executing on the user device, additional physiological data collected from the user via the wearable device, collected from an additional user via an additional wearable device, or both, wherein the satisfaction of the trigger event is based at least in part on the additional physiological data.

4. The wearable device of claim 1, further comprising:
receiving, from one or more additional applications executing on the user device, additional data associated with environmental surroundings of the user, a schedule of the user, a navigational destination of the user, or any combination thereof, wherein the satisfaction of the trigger event is based at least in part on the additional data.

5. The wearable device of claim 1, wherein the one or more learning objectives comprise an objective for managing stress, the method further comprising:
receiving, at the application executing on the user device, additional physiological data collected from the user via the wearable device, wherein the additional physiological data comprises heart rate data, heart rate variability data, temperature data, or any combination thereof; and
determining a stress metric associated with the user based at least in part on the heart rate data, the heart rate variability data, temperature data, or any combination thereof, the stress metric associated with a relative level of stress or relaxation experienced by the user, wherein identifying the satisfaction of the trigger event is based at least in part on the stress metric satisfying a threshold stress level.

6. The wearable device of claim 1, wherein the one or more learning objectives comprise an objective to recognize or lower blood pressure, the method further comprising:
receiving, at the application executing on the user device, additional physiological data collected from the user via the wearable device; and
determining a blood pressure metric associated with the user based at least in part on the additional physiological data, wherein identifying the satisfaction of the trigger event is based at least in part on the blood pressure metric being greater than or equal to a threshold blood pressure metric.

7. The wearable device of claim 1, wherein the one or more learning objectives comprise an objective to match a running or walking cadence of the user to a heart rate of the user, the method further comprising:
receiving, at the application executing on the user device, additional physiological data collected from the user via the wearable device, wherein the additional physiological data comprises heart rate data and motion data;
determining the running or walking cadence of the user based at least in part on the motion data;
determining the heart rate of the user based at least in part on the heart rate data; and
determining that a difference between the heart rate and the running or walking cadence satisfies a threshold difference, wherein identifying the satisfaction of the trigger event is based at least in part on the difference satisfying the threshold difference.

8. The wearable device of claim 1, wherein the one or more learning objectives comprise an objective to adjust or maintain one or more physiological parameters of the additional user, the method further comprising:
receiving, at the application executing on the user device, additional physiological data collected from the additional user via an additional wearable device, wherein the satisfaction of the trigger event is based at least in part on the additional physiological data, and wherein monitoring the physiological data comprises monitoring the physiological data collected from the additional user.

9. The wearable device of claim 1, wherein the tactile or audible feedback provided by the wearable device comprises one or more tactile vibrations, a temperature change of a surface of the wearable device, a pressure change exerted by the wearable device on a tissue of the user, or any combination thereof.

10. The wearable device of claim 1, wherein the one or more feedback instructions are executable by the wearable device to provide the tactile or audible feedback to the user subconsciously in accordance with one or more characteristics of the tactile or audible feedback, wherein the one or more characteristics of the tactile or audible feedback comprise a magnitude, a volume, a cadence, or any combination thereof.

11. The wearable device of claim 1, wherein the one or more parameters of the learning feedback loop comprise a magnitude or volume of the tactile or audible feedback, a threshold associated with the trigger event, a feedback type of the learning feedback loop, or any combination thereof.

12. The wearable device of claim 1, wherein the physiological data is collected from the user via the wearable device, and wherein the one or more learning objectives comprise an objective to reduce or manage stress, an objective to recognize or lower blood pressure, an objective to match a running or walking cadence to a heart rate, an objective to improve engagement or relaxation for other users, or any combination thereof.

13. The method of claim 1, wherein the wearable device comprises a wearable ring device.

14. An apparatus, comprising:
one or more memories storing processor-executable code; and
one or more processors coupled with the one or more memories and individually or collectively operable to execute the code to cause the apparatus to:
identify, at an application executing on a user device, a satisfaction of a trigger event for providing tactile or audible feedback to a user via a wearable device;
communicate to the wearable device, based at least in part on the satisfaction of the trigger event, one or more feedback instructions that are executable by the wearable device to provide the tactile or audible feedback to the user via the wearable device and in accordance with a learning feedback loop associated with the user, wherein the learning feedback loop is configurable to train the user, via the tactile or audible feedback, to learn or perform one or more learning objectives associated with the trigger event;
monitor physiological data collected from the user, an additional user, or both, based at least in part on communicating the one or more feedback instructions; and
selectively modify one or more parameters of the learning feedback loop based at least in part on an evaluation of the physiological data relative to the one or more learning objectives.

15. The apparatus of claim 14, wherein the one or more processors are individually or collectively further operable to execute the code to cause the apparatus to:
receive, via a user device associated with the user, a user input indicating the one or more learning objectives, wherein identifying the satisfaction of the trigger event is based at least in part on receiving the user input.

16. The apparatus of claim 14, wherein the one or more processors are individually or collectively further operable to execute the code to cause the apparatus to:
receive, at the application executing on the user device, additional physiological data collected from the user via the wearable device, collected from an additional user via an additional wearable device, or both, wherein the satisfaction of the trigger event is based at least in part on the additional physiological data.

17. The apparatus of claim 14, wherein the one or more processors are individually or collectively further operable to execute the code to cause the apparatus to:
receive, from one or more additional applications executing on the user device, additional data associated with environmental surroundings of the user, a schedule of the user, a navigational destination of the user, or any combination thereof, wherein the satisfaction of the trigger event is based at least in part on the additional data.

18. The apparatus of claim 14, wherein the one or more learning objectives comprise an objective for managing stress, and wherein the one or more processors are individually or collectively further operable to execute the code to cause the apparatus to:
receive, at the application executing on the user device, additional physiological data collected from the user via the wearable device, wherein the additional physiological data comprises heart rate data, heart rate variability data, temperature data, or any combination thereof; and
determine a stress metric associated with the user based at least in part on the heart rate data, the heart rate variability data, temperature data, or any combination thereof, the stress metric associated with a relative level of stress or relaxation experienced by the user, wherein identifying the satisfaction of the trigger event is based at least in part on the stress metric satisfying a threshold stress level.

19. The apparatus of claim 14, wherein the one or more learning objectives comprise an objective to recognize or lower blood pressure, and wherein the one or more processors are individually or collectively further operable to execute the code to cause the apparatus to:
receive, at the application executing on the user device, additional physiological data collected from the user via the wearable device; and
determine a blood pressure metric associated with the user based at least in part on the additional physiological data, wherein identifying the satisfaction of the trigger event is based at least in part on the blood pressure metric being greater than or equal to a threshold blood pressure metric.

20. An apparatus, comprising:
means for identifying, at an application executing on a user device, a satisfaction of a trigger event for providing tactile or audible feedback to a user via a wearable device;
means for communicating to the wearable device, based at least in part on the satisfaction of the trigger event, one or more feedback instructions that are executable by the wearable device to provide the tactile or audible feedback to the user via the wearable device and in accordance with a learning feedback loop associated with the user, wherein the learning feedback loop is configurable to train the user, via the tactile or audible feedback, to learn or perform one or more learning objectives associated with the trigger event;
means for monitoring physiological data collected from the user, an additional user, or both, based at least in part on communicating the one or more feedback instructions; and
means for selectively modifying one or more parameters of the learning feedback loop based at least in part on an evaluation of the physiological data relative to the one or more learning objectives.
